(19) United States
(12) Patent Application Publication
(10) Pub. No.: US 2024/0164891 A1
(43) Pub. Date: May 23, 2024

(54) OSSICULAR PROSTHESIS

(71) Applicant: MED-EL ELEKTROMEDIZINISCHE GERÄTE GMBH, Innsbruck (AT)

(72) Inventors: Uwe Steinhardt, Hirrlingen (DE); Clemens Kuen, Imst (AT); Stefan Rainer, Grinzens (AT); Lukas Mair, Schwaz (AT); Jörg Hausch, Ofterdingen (DE)

(21) Appl. No.: 18/283,703

(22) PCT Filed: Mar. 24, 2022
(86) PCT No.: PCT/EP22/57793

§ 371 Date: Sep. 22, 2023

(30) Foreign Application Priority Data
Mar. 30, 2021 (DE) 10 2021 107 955.1

(51) Int. Cl. A61F 2/18; A61L 27/04

(52) U.S. Cl. CPC A61F 2/18; A61L 27/04; A61F 2002/183; A61F 2220/0033

(57) ABSTRACT

The invention proposes pre-load a ball joint (4) of an auditory ossicle prosthesis (1) with a disk-shaped pre-loading element (11) in a longitudinal direction of a shaft (7) of the auditory ossicle prosthesis (1), whereby an almost loss-free vibration transmission is achieved.

The invention relates to an auditory ossicle prosthesis with the embodiments of the preamble of claim 1.

Auditory ossicle prostheses serve to replace one or more auditory ossicles in the human middle ear and, instead of the replaced auditory ossicle or ossicles, transmit vibrations generated by sound waves from the eardrum to the oval window of the inner ear.

The patent application US 2007/0083263 A1 discloses an ossicle prosthesis with a head plate that is arranged inside an eardrum, a foot element that is attached to an ossicle or an inner ear, a shaft that connects the head plate and the foot element, and a ball joint, that movably connects the head plate to the shaft. A ball socket of the ball joint of the known auditory ossicle prosthesis is made from an elastic plastic.

The object of the invention is an auditory ossicle prosthesis with a ball joint with good transmission of mechanical vibrations from an eardrum to an auditory ossicle of a human middle ear or to an oval window of a human inner ear. A further object of the invention is to follow post-operative movements caused by scar tissue and this in turn caused by the healing process taking place on the reconstructed eardrum, and thus to avoid pressure peaks that could negatively affect the acoustomechanical signal transmission.

The auditory ossicle prosthesis according to the invention with the embodiments of claim 1 has a head element, a foot element, a ball joint that movably connects the head element and the foot element, and a shaft that connects the head element to the foot element, the ball joint being on the head element, foot element or can be arranged at any point of the shaft and is preferably arranged on the head element.

The head element is a fastening element that is arranged on the inside of an eardrum in a human middle ear in such a way that it transmits mechanical vibrations of the eardrum caused by sound to the ossicle prosthesis, but in turn allows macro movements caused by movements in the eardrum. In this context, the term eardrum is understood to mean both an existing, partially existing and an artificially complete or partially reconstructed eardrum as well as a partially existing and the remaining part reconstructed eardrum. Reconstruction includes any form of production of a vibratory membrane, for example with body tissue or an artificially created vibration membrane for mechanical sound transmission. This includes an arrangement or attachment of the head element to an auditory ossicle or to a vibration generator, which generates sound-generating vibrations in the ear instead of the eardrum.

The foot element is a fastening element that is arranged on an auditory ossicle, in particular on a stapes in the human middle ear or on the oval window of a human inner ear, in such a way that it transmits mechanical vibrations from the auditory ossicle prosthesis to the auditory ossicle or the oval window. This includes an arrangement or fastening attachment of the foot element to an auditory ossicle other than the stapes or elsewhere in a human ear.

The ball joint has a ball receptacle and a ball head, which is arranged in the ball receptacle so that it can pivot two-dimensionally about a center point of the ball head. The ball head has the shaft protruding from it, which is generally an element for connecting the ball head to the foot element or the head element. The ball and socket joint adjusts an angular position of the head element to a position of the eardrum in relation to the ossicular prosthesis. In order to accommodate the ball head in the ball receptacle without play, the invention provides that the ball receptacle holds the ball head in a position of use of the shaft with a pre-load in both a radial and an axial direction of the shaft. The position of use is a position that the shaft assumes in relation to the ball receptacle when the ossicle prosthesis is implanted in a human ear. Instead of the position of use, a position of the shaft perpendicular to the eardrum or a central pivoting position of the shaft can also be selected. Another definition of the same situation is a play-free reception of the ball head in the ball receptacle with a pre-load both parallel to a tympanic membrane plane and perpendicular to the tympanic membrane plane. The tympanic membrane plane is an imaginary tangential plane of the tympanic membrane at a point where the head element of the auditory ossicle prosthesis is arranged. It can be assumed that the head element is arranged on the eardrum as intended. As long as the shaft of the ball head is aligned perpendicular to the plane of the eardrum, both definitions are equivalent. If the shaft has an angle perpendicular to the eardrum plane, the two definitions differ by this angle, which can be understood as a tolerance for the directions of the pre-load forces.

The mechanical vibrations of the eardrum generated by sound are transmitted in a longitudinal direction of the shaft. For a good transmission of the vibrations from the ball receptacle to the ball head of the ball joint of the auditory ossicle prosthesis according to the invention, the ball head in the ball receptacle must be arranged in an inelastic arrangement that is as play-free as possible Longitudinal direction, i.e. in the axial direction of the shaft, is necessary, which is achieved by pre-loading the ball head perpendicular to the drum filing plane or in the axial direction of the shaft in the ball receptacle. As a result, the mechanical vibrations are not at all dampened, or at most only slightly dampened.

In one embodiment of the invention provides an elastic pre-load element on a side opposite the shaft in relation to the ball head, which acts on the ball head in the direction of the shaft. In this embodiment of the invention a defined position of the ball head in the ball receptacle is achieved and causes good vibration transmission in one direction of vibration.

A simple version, which enables a compact ball receptacle, a compact ball joint and a compact head element, provides a spring washer as a pre-loading element, which is arranged elastically perpendicular to a surface defined by it and which is arranged tangentially to the ball head. The spring washer can be circular, round, square, strip-shaped and in general of any shape; it can be hole-free or have a center hole or one or more other holes. For example, a spring washer with a slot and preferably several slots arranged in a star shape is also possible. Due to its surface shape, the spring washer can be arranged tangentially to the ball head on or in the ball receptacle to save space. Through openings such as the hole or center hole or one or more slots, the spring properties of the spring washer can be influenced, in particular its spring hardness can be changed. A center hole in the spring washer or star-shaped, in particular radially arranged slots or generally slots that meet in a center of the spring washer center the ball head of the ball joint in the ball receptacle of the auditory ossicle prosthesis.

In one embodiment of the invention provides for the ball receptacle to be designed as a snap device into which the ball head is snapped in a manner similar to a push button. In one embodiment of the invention, the ball receptacle has snap elements which are arranged around the ball head distributed over a circumference and between which the ball head is snapped. The snap elements can be evenly or unevenly distributed in the circumferential direction. be ordered and/or they can, for example, be finger-shaped or strip-shaped and resilient radially to the ball head. The snap elements rest on the ball head with a pre-load acting parallel to the eardrum or radially to the shaft in the position of use and ensure that the ball head is held in the ball receptacle without play parallel to the eardrum or radially to the shaft in the position of use. Another advantage of designing the ball receptacle as a snap device is that the ball joint is easy to assemble.

In a preferred embodiment of the invention, the ball receptacle and the ball head are made of metal or ceramic and lie directly against each other without the interposition of another element that could dampen or otherwise impair the vibration transmission from the ball receptacle to the ball head or vice versa. This embodiment of the invention causes good vibration transmission with low losses. The ball head and the ball receptacle can be made of titanium alloys or stainless steel, for example.

All embodiments mentioned in the description and/or shown in the drawing can be implemented individually or in any combination in embodiments of the invention. Embodiments of the invention that do not have all, but only some, of the embodiments of a claim, including the independent claim, are possible.

The invention is explained in more detail below using an exemplary embodiment shown in the drawing. It shows:

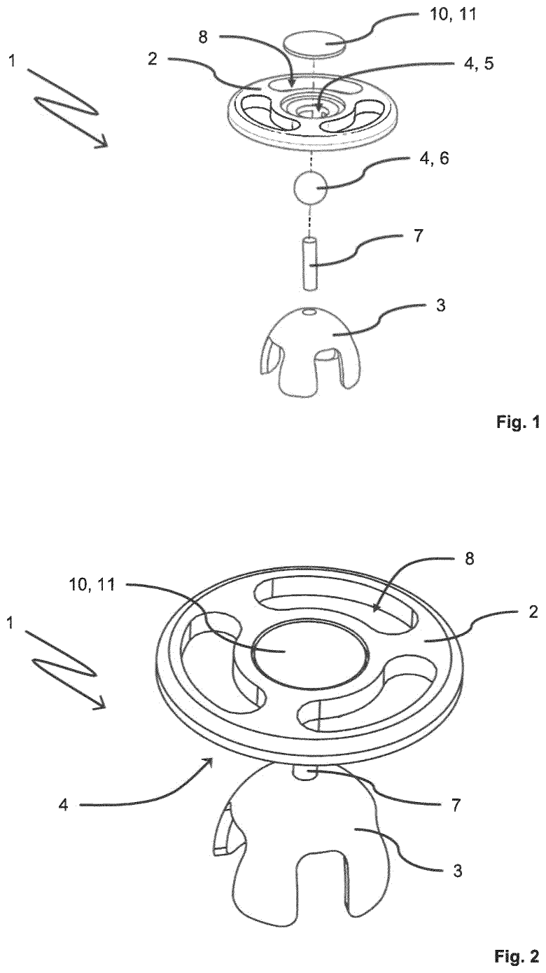

FIG. 1 shows an auditory ossicle prosthesis according to the invention in a perspective exploded view;

FIG. 2 shows the auditory ossicle prosthesis from FIG. 1 in an assembled state in a perspective view;

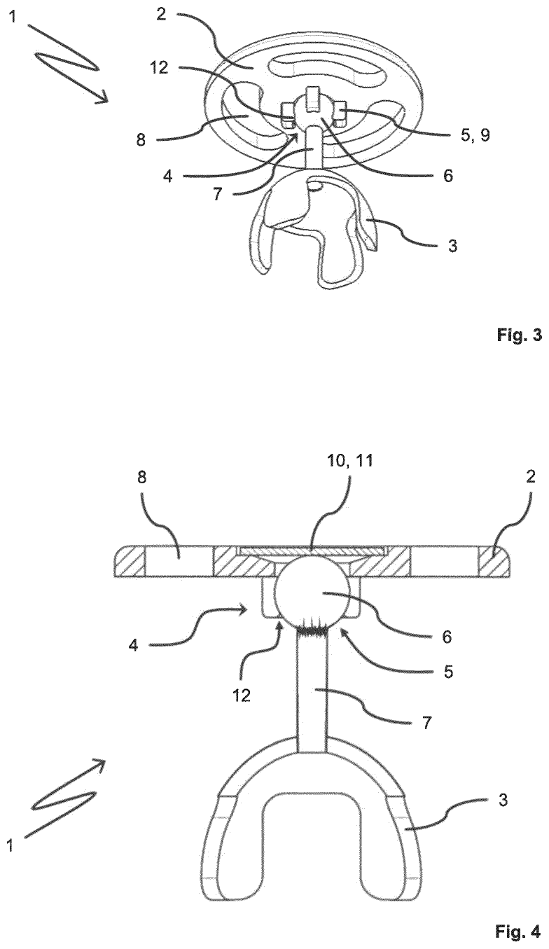

FIG. 3 shows the auditory ossicle prosthesis from FIG. 2 with a different viewing direction;

FIG. 4 shows an axial section of a head element and a ball head of the auditory ossicle prosthesis from FIGS. 1 and 2 in an enlarged representation;

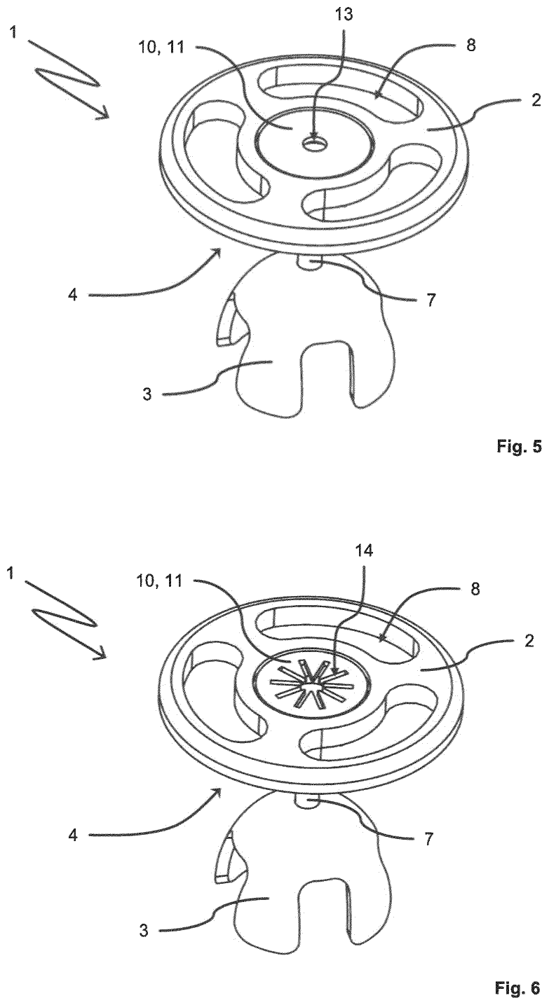

FIGS. 5 and 6 show modifications of the auditory ossicle prosthesis from FIGS. 1 to 4 according to the invention in perspective representations corresponding to figures.

Basically, the same parts in the figures are provided with the same reference numbers.

The auditory ossicle prosthesis 1 according to the invention shown in FIGS. 1 to 4 is intended to replace one or more auditory ossicles in a human ear. It has a head element 2, which is intended to be arranged on the inside of an eardrum of the ear, and a foot element 3, which is intended to be attached to an auditory ossicle, in particular to a stapes or an oval window in the middle ear or inner ear of a human being. The auditory ossicle prosthesis 1 transmits vibrations of the eardrum generated by sound waves to the stapes or the element of the ear to which the foot element 3 is attached. In this context, an eardrum is to be understood as meaning both an existing, partially existing and an artificially complete or partially reconstructed eardrum, as well as a partially existing and the remaining part reconstructed. The reconstruction includes any form of manufacturing an vibrating membrane, for example with body tissue or an artificially created vibrating membrane for mechanical sound transmission. The head element can also be designed to be attached to a vibration generator that generates vibrations that generate sound in the ear (not shown).

The auditory ossicle prosthesis 1 comprises the head element 2, the foot element 3, a ball joint 4 with a ball receptacle 5, a ball head 6 and a shaft 7. In the exemplary embodiment, the ball receptacle 5 is part of the head element 2. The shaft 7 protrudes radially from the ball head 6; in the exemplary embodiment it is welded to the ball head 6, but it can also be rigidly connected to the ball head 6 in some other way, for example by being screwed in. The ball joint 4 connects the shaft 7 two-dimensionally pivotable about a center point of the ball head 6 with the head element 2. The shaft 7 rigidly connects the foot element 3 to the ball head 6. As a result, the head element 2 and the foot element 3 can be pivoted in two dimensions about the center point of the ball head 6 and are connected to one another at a fixed distance from one another.

In the illustrated and described exemplary embodiment of the invention, the head element 2 of the auditory ossicle prosthesis 1 is a circular, flat disk with a central hole and elongated holes 8 extending in the shape of a circular arc in the circumferential direction. Other configurations of the head element 2 are possible, whereby configurations designed to be arranged on a vibration generator instead of on the inside of the eardrum are also possible (not shown).

The foot element 3 of the auditory ossicle prosthesis 1 is bell-shaped in the exemplary embodiment, although other designs are possible.

In the exemplary embodiment, the shaft 7 is a cylindrical pin, at the end of which the foot element 3 is welded to the end remote from the ball head 6 or is rigidly fixated in some other way.

The head element 2 of the auditory ossicle prosthesis 1 has the ball receptacle 5 in which the ball head 6 rests in two dimensions so that it can be pivoted about its center point. The ball socket 5 and the ball head 6 form the ball joint 4.

In the exemplary embodiment, the ball receptacle 5 is designed as a snap-in device into which the ball head 6 is snapped like a push button. In the exemplary embodiment, the ball receptacle 5 has four finger-shaped snap elements 9 which are arranged around the center hole of the head element 2 and protrude from one side of the head element 2. A distance between the snap elements 9 is smaller than a diameter of the ball head 6, so that the snap elements 9 bear against the ball head 6 with an elastic spring force, which is directed in the direction of the center point of the ball head 6. The elastic spring force of the snap elements 9 can also be understood as a pre-load parallel to the head element 2. The disk-shaped head element 2 in the exemplary embodiment defines an eardrum plane, which is a tangential plane of an eardrum at a point at which the head element 2 is arranged on the eardrum when the head element 2 is arranged on the eardrum in the intended manner. The pre-load of the snap elements 9 also acts radially to the shaft 7 when it is aligned perpendicular to the head element 2, which can be viewed as the position of use of the shaft 7. The usage position of the shaft 7 is the position that the shaft 7 assumes when the ossicular prosthesis 1 is implanted in an ear. Ends of the snap elements 9 remote from the head element 2 are designed to stand somewhat hookshaped inwards towards the ball head 6, so that they reach under the ball head 6 on a side facing away from the head element 2 and hold it in the ball receptacle 5.

The number of four snap elements 9 is not mandatory for the invention; fewer or more snap elements 9 can also be provided and the snap elements 9 can also be arranged unevenly instead of uniformly distributed in the circumferential direction of the ball head 6 (not shown).

In the center hole of the head element 2, a spring washer 10 which is elastic perpendicular to its surface is arranged as a pre-loading element 11, which in the exemplary embodiment is connected to the head element 2 by welding, but can also be fixed to the head element 2 or the ball receptacle 5 in a different way. The preloading element 11 is arranged tangentially to the ball head 6 on a side of the ball head 6 facing away from the shaft 7; it closes the ball receptacle 5 on a side of the ball head 6 opposite the shaft 7. A distance between the spring washer 10 forming the pre-loading element 11 and the hook shaped ends 12 of the snap elements 9, which engage under the ball head 6 on the side facing away from the pre-loading element 11, are chosen to be so small that the ball head 6 elastically deforms the pre-loading element 11, whereby the pre-loading element 11 exerts an elastic force perpendicular to the head element 2 and in the longitudinal direction of the shaft 7 on the ball head 6. This elastic force can be understood as a pre-loading perpendicular to the head element 2 or perpendicular to the plane of the eardrum and in the longitudinal direction of the shaft 7. The pre-load that the pre-loading element 11 exerts on the ball head 6 holds the ball head 6 without play perpendicular to the head element 2 and in the longitudinal direction of the shaft 7, i.e. in the direction in which vibrations of the eardrum via the auditory ossicle prosthesis 1 onto the stapes, other ossicles or the oval window. This freedom from play in the ball joint 4 of the auditory ossicle prosthesis 1 according to the invention causes an almost loss-free vibration transmission from the head element 2 to the foot element 3 or in any case in the ball joint 4 of the auditory ossicle prosthesis 1.

The components of the auditory ossicle prosthesis 1, in particular the ball receptacle 5 and the ball head 6, are made of metal or ceramic, for example stainless steel or a titanium alloy. The ball head 6 lies directly and without any intermediate element that could dampen the vibration transmission or worsen it in any other way, in the ball receptacle 5 or between the snap elements 9 and the spring washer 10 forming the pre-loading element 11, which ensures good and largely loss-free vibration transmission from the head element 2 on the ball head 6 is achieved.

In the embodiment of an auditory ossicle prosthesis 1 according to the invention shown in FIG. 5, the spring washer 10 forming the pre-loading element 11 has a center hole 13 which centers the spring washer 10 on the ball head 6 during welding.

In the embodiment of an auditory ossicle prosthesis 1 according to the invention shown in FIG. 6, the spring washer forming the pre-loading element 11 has 10 radial slots 14, which meet in a center of the spring washer 10 and center the spring element on the ball head 6 during welding.

The center hole 13 and the slots 14 influence the spring properties of the spring washer 10, in particular a spring hardness of the spring washer 10, through their shape, size and arrangement.

1. Auditory ossicle prosthesis, comprising:
a head element configured to be arranged on an eardrum,
a foot element configured to be arranged on an auditory ossicle in the human middle ear or on the oval window of a human inner ear, and
a Ball joint, which connects the head element and the foot element in an articulated manner, the ball joint having a ball receptacle and a ball head configured to be pivoted two-dimensionally about its center in the ball receptacle wherein the ball receptacle holds the ball head without play with a pre-load perpendicular to the plane of the eardrum or in a longitudinal direction of the shaft in the position of use of the shaft.

2. Auditory ossicle prosthesis according to claim 1, wherein the ball receptacle has an elastic pre-loading element on a side of the ball head opposite the shaft which acts on the ball head in the direction of the shaft.

3. Auditory ossicle prosthesis according to claim 2, wherein the pre-loading element is a spring washer arranged tangentially to the ball head.

4. Auditory ossicle prosthesis according to claim 3, wherein the spring washer has a hole, a central hole, a slot, or slots arranged in a star shape.

5. Auditory ossicle prosthesis according to claim 1, wherein the ball receptacle has snap elements which are distributed over a circumference, between which the ball head is snapped and which are parallel to the pre-load to the eardrum level or radially to the shaft in the use position of the shaft on the ball head.

6. Auditory ossicle prosthesis according to claim 1, wherein the ball receptacle and the ball head are made of metal or ceramic and lie directly against one another.

7. Auditory ossicle prosthesis according to claim 2, wherein the ball receptacle has snap elements which are distributed over a circumference, between which the ball head is snapped and which are parallel to the pre-load to the eardrum level or radially to the shaft in the use position of the shaft on the ball head.

8. Auditory ossicle prosthesis according to claim 3, wherein the ball receptacle has snap elements which are distributed over a circumference, between which the ball head is snapped and which are parallel to the pre-load to the eardrum level or radially to the shaft in the use position of the shaft on the ball head.

9. Auditory ossicle prosthesis according to claim 2, wherein the ball receptacle and the ball head are made of metal or ceramic and lie directly against one another.

10. Auditory ossicle prosthesis according to claim 3, wherein the ball receptacle and the ball head are made of metal or ceramic and lie directly against one another.

11. Auditory ossicle prosthesis according to claim 4, wherein the ball receptacle and the ball head are made of metal or ceramic and lie directly against one another.

12. Auditory ossicle prosthesis according to claim 5, wherein the ball receptacle and the ball head are made of metal or ceramic and lie directly against one another.
